(19) United States
(12) Patent Application Publication
(10) Pub. No.: US 2022/0223258 A1
(43) Pub. Date: Jul. 14, 2022

(54) AUTOMATIC DELIVERY OF PERSONALIZED MESSAGES

(71) Applicant: The Penn State Research Foundation, University Park, PA (US)

(72) Inventors: Timothy R. Brick, State College, PA (US); Zita Oravecz, State College, PA (US); James Patrick Mundie, Port Matilda, PA (US)

(21) Appl. No.: 17/608,066

(22) PCT Filed: May 1, 2020
(86) PCT No.: PCT/US2020/030955

§ 371 Date: Nov. 1, 2021

Related U.S. Application Data
(60) Provisional application No. 62/842,221, filed on May 2, 2019.

(51) Int. Cl. G16H 20/70; H04L 51/216; H04L 51/21; H04L 51/42; H04L 51/046

(52) U.S. Cl. CPC G16H 20/70; H04L 51/216; H04L 51/046; H04L 51/42; H04L 51/21

(57) ABSTRACT

Messages are automatically generated on a user device. Physiologic data generated from the sensing of one or more physiological parameters of a user is received. User-entered data for the user is received. Social-context data is received describing one or more other user is within social communication with the user. A determination is generated from i) the physiologic data, ii) the user-entered data, iii) the social-context data, iv) historical, archival, or prerecorded data that records at least one profile of historical data that an automated message should be sent to one or more user devices. The automated message is sent.

### CROSS-REFERENCE TO RELATED APPLICATIONS

This application claims priority to U.S. Application Ser. No. 62/842,221, filed on May 2, 2019. The disclosure of the prior application is considered part of the disclosure of this application, and is incorporated in its entirety into this application.

### TECHNICAL FIELD

This document describes technology for the automated sensing of user data using one or more sensors.

### BACKGROUND

Mental health is a level of psychological well-being or an absence of mental illness—the state of someone who is functioning at a satisfactory level of emotional and behavioral adjustment. From the perspectives of positive psychology or of holism, mental health may include an individual's ability to enjoy life, and to create a balance between life activities and efforts to achieve psychological resilience.

### SUMMARY

This document describes technology that can be used to passively collect data about a user's social context. This social-context data can be continuously generated and combined with automatically sensed physiological data and the reception of user-input data in order to drive decisions about when and if messages should be delivered, and about the content of the messages. For example, the technology may be used to automatically provide interventions in the form of text communications in response to a user entering a particular emotional state (e.g., a stress state, a craving state, or a high risk of a spousal dispute.) This may be of particular value when the state may be detrimental to a user's health, such as a user with a stress disorder entering a stress state, a user with and addiction disorder entering a craving state, etc.

In order to do so, technology described in this document can be used to integrate data about social interaction, physiology, and overall well-being into a unified platform for social and behavioral wellness. For example, a smartphone app that uses commodity wearable hardware can be used to provide actionable feedback to a user based on physiological changes enacted by their day-to-day behaviors, especially in the context of social interactions.

This technology takes into account contextual factors, specifically social contexts that can help improve the user's experience. For example, do users show increases in physiological stress when they spend time with their families or a possible decrease in stress when they spend time alone? The value of these insights can be augmented by integrating multiple streams of data that can be gleaned from both the environment and other users in the environment. The data is given in the context of the user's daily social situation. This will give further insight into the interactions with family members, coworkers, spouses, etc.

In some implementations, a method can be used for the automated generation of messages on a user device. The method can include receiving physiologic data generated from the sensing of one or more physiological parameters of a user. The method can further include receiving user-entered data for the user. The method can further include receiving social-context data describing one or more other user is within social communication with the user. The method can further include generating a determination, from i) the physiologic data, ii) the user-entered data, iii) the social-context data, iv) historical, archival, or prerecorded data about the individual or others that records at least one profile of historical data that an automated message should be sent to one or more user devices. The method can further include sending, responsive to the generating the determination and to the one or more user devices, the automated message. Other technology including systems, devices, computer-readable media, and software can be used for the automated generation of messages on a user device.

Implementations can include some, all, or none of the following features. Generating the determination that the automated message should be sent to a user device comprises initially using at least one of the group consisting of i) the physiologic data, ii) the user-entered data, and iii) the social-context data to generate an initial determination that the user, dyad, or group is in a particular state; and after generating the initial determination, using at least one of the group consisting of i) the physiologic data, ii) the user-entered data, and iii) the social-context data to confirm the initial the determination. Generating the determination that the automated message should be sent to a user device comprises applying i) the physiologic data, ii) the user-entered data, and iii) the social-context data to a predictor that generates a prediction of the user's particular state. The physiologic data comprises readings of at least one of the group consisting of cardiac action, respiratory action, gross body-motion, body temperature, skin electrical properties, functional or structural brain signals. The social-context data is generated from location data comprises at least one of the group consisting of Global Positioning System (GPS) readings, geographic coordinates, data-network based geopositioning readings, and a proximity measure to a physical device. The social-context data is generated by identifying at least one of the group consisting of a location of other users, a social context of at least one other user, and a location of devices of other users. Identifying at least one of the group consisting of the location of other users and the location of devices of other users comprises at least one of the group consisting of gathering data from a Bluetooth data connection, gathering data from a Zigbee data connection, gathering data from a Near Field Communication (NFC) data connection, gathering data from an audio sensor, gathering data from a Radio Frequency Identification (RFID) sensor, and gathering data from a sensor. The sensor is at least one of the group consisting of a microphone, a camera, a depth sensor, a thermal sensor, a vibration sensor, an appliance activity sensor, a standalone broadcast beacon and a receiver, a weight and pressure sensors, a light detecting and ranging (LIDAR) sensor, a sonar sensor, an ultrasonic sensor and a radio reflectance sensor. Sending the automated message comprises at least one of the group consisting of sending the automated message to a device associated with the user, sending the automated message to another user within the social-context, sending the automated message to another user not within the social-context, ending the automated message to a device not associated with a user within the social-context, and storing the automated message for display at a later time. Generating a determination that an automated message should be send to a user device further comprises using v) other theory-, evidence-, or other model-based rules, and vi) with human approval or adjustment. Sending of the automated message to a long-term report for use by another user

Some implementations of the technology described in this document can be used to realize one or more advantages. The technology of medical interventions can be improved. For example, interventions can be provided to a user in a time, place, and format that is useful for delivering behavior-based interventions to a user. Because a clinician (or other expert) can generate rules ahead of time, interventions can be provided with or without the immediate attention to of a clinician. By incorporating a social context determined from sensor data, the social context can be included in determinations to provide interventions, which is an advantage over systems that cannot utilize social context.

### DESCRIPTION OF DRAWINGS

FIG. 1 shows an example system for the automatic generation of messages on a user device.

FIG. 2 shows a flowchart of an example process for generating and delivering a message on a user device.

FIG. 3 is a schematic diagram that shows an example of a computing system.

Like reference symbols in various drawings indicate like elements.

### DETAILED DESCRIPTION

Technology described in this document can be used to integrate data about a user's social setting that automatically generates messages to the user. In some implementations, this can include providing the user with interventions on their mobile device in a way that supports their mental health (or other health) treatments and their lifestyle/behavior goals.

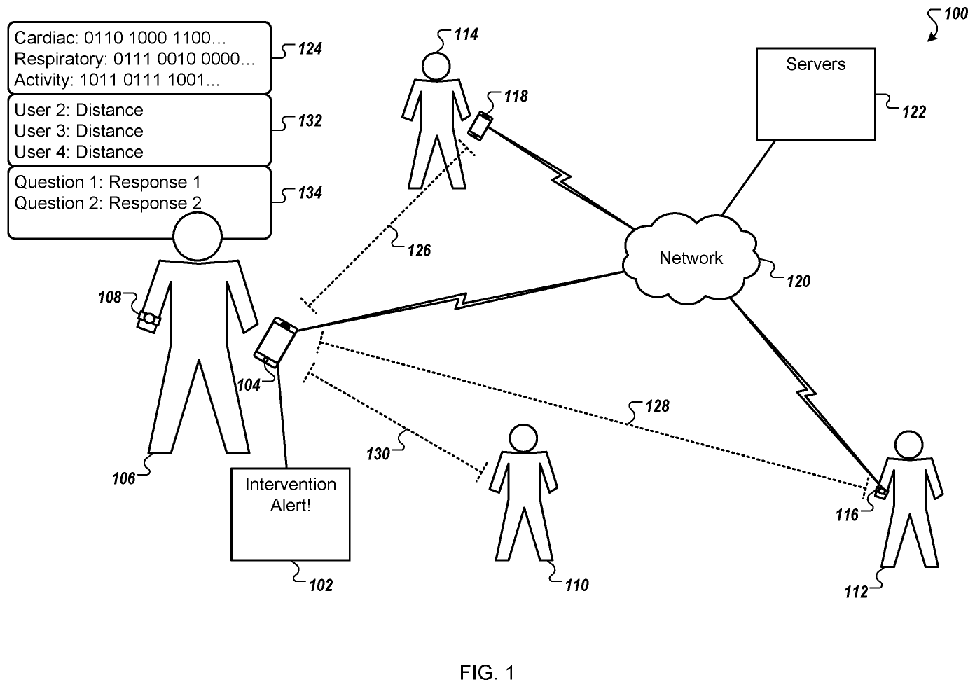

FIG. 1 shows an example system 100 for the automatic generation of messages 102 on a user device 104. In the system 100, a user 106 is carrying the user device 104 (e.g., a cellular telephone) and another user device 108 (e.g., a wearable activity tracker.) The system 100 includes other users 110, 112, and 114. As shown here, the user 112 is wearing a user device 116 and the user 114 is carrying a user device 118. A network 120 connects the user devices 104, 116, 118 and other computing device such as a server 122.

In this example, the system 100 is configured to use data about the user 106 to automatically generate messages for the user. For example, the user may be receiving healthcare or wellness services from a clinic or application, and the user device 104 can be configured to provide messages for these services to the user 106. These message may include messages or other interventions that guide the user 106 through treatments designed to help the user in some way, for example as treatment for a health condition. The system 100, or another similar system, can also be used to assist the user 106 in reaching behavior-based and lifestyle goals. For example, to help an athlete consume sufficient protein according to a feeding schedule, the system 100 can identify social contexts within various feeding time windows in which the athlete can comfortably have a protein shake without feeling embarrassment or socially uncomfortable. For example, the athlete may be comfortable having the protein shake around friends and family, but not around an employer or teacher, and the system 100 may remind the athlete when around friends and family, but not around employers or teachers.

The user's 106 physiologic parameters (e.g., cardiac action, respiratory action, gross body action, body temperature, skin electrical properties) can be monitored by the devices 104 and/or 108, working alone or in combination. From these parameters, physiologic data 124 can be generated, comprising computer-readable data representing some aspect of the physiologic parameters. For example, the physiologic data 124 may include a stream of computer data (e.g., binary digits) that represent heart rate, heart-rate variability, cardiac-action classification (e.g., normal, out-of-normal,) etc.

Location data for the user 106 can be generated by one or both of the user devices 104 and 108. In some cases, the location data includes geographic data, that is, data specifying where the user is on Earth, perhaps in longitude and latitude terms. In some cases, the location data includes relational data. For example, the user device 104 connect with a user device 116 (e.g., via Bluetooth, Zigbee, Near Field Communication (NFC,) etc.) and determine the distance between the user devices 104 and 116. From this, the distance between the users 112 and 106 can be determined. In another example, the location of the user device 104 can be determined by examining the data networks available to the user device 104. For example, if the user device 104 is within range of a wireless network having a known location, the approximate location of the user device 104 can be found.

The social context of the user 106 can be determined. In general, the social context of the user 106 is a listing of other users and environmental factors that are likely to socially influence the user 106.

For example, based on the location data of the user devices 104, 108, 116, and 118, the proximity of the users 112 and 114 to the user 106 can be found. If this proximity is low (e.g., less than a threshold distance,) it can be determined that the users 112 and/or 114 are within the social context of the user 106. In addition, users may be sensed in a more direct manner in order to determine social context. For example, a sensor may sense the location of the user 110 directly, even though in this case the user 110 is not using any user devices 110. In some cases, a home automation-hub may use one or more sensors (e.g., microphones, cameras, depth sensors, thermal sensors, vibration sensors, appliance activity sensors, standalone broadcast beacons and receivers, weight or pressure sensors, lidar, sonar, ultrasonic sensors and radio reflectance sensors) to interact with users and determine where a user is within the home. In another case, a security camera in a store, treatment facility, etc. may identify the location of shoppers or patients within a building. Based on relative locations of the users 106, 110, 112, and 114, physical distances 126, 128, and 130 may be measured. Based on these measured distances 126, 128, and 130, in combination with other data (e.g., location data for other users or other physical landmarks) the social context of the user may be recorded 132.

User-entered data 134. For example, and application on the user device 104 may present to the user an interface asking the user about their emotional state or physical state, about the environment, etc. The questions and answers can be recorded 134. This interface may take the form of an electronic device with a touch-screen, verbal input, etc. that presents a user with an input. This input may be text based, verbal, or other types of checklists of feelings, appraisals, behaviors, perceptions, recall of past behaviors, and tendencies to any of the above, sliders, multidimensional surfaces or volumes or radio buttons indicating the quantity of these, virtual clocks or other interactive or non-interactive skeuomorphic or virtualized objects, or via recording and analysis (manual or automated) of audio, video, depth, text, or other multimedia signals, interactive games, tests, measurement tools or assessments, adaptive combinations of these, or data derived from these, their histories, and other data sources.

In response to the physiologic data 124, the user entered data 134, and the social-context data 132, a user device 104 can present an intervention alert 102. Generally speaking, the intervention alert 102 can include instructions or requests for the user that are part of an intervention of some type.

In one example, the user 106 is a patient that has been discharged from a substance-abuse rehabilitation facility. As part of the user's 106 treatment plan, the user 106 is enrolled in a program where the user's 106 mobile and wearables devices 104 record social context and biometric data in order to provide context relevant interventions to the user 106 when the user 106 is in a craving state. That is, the user's 106 technology advantageously helps the user 106 stick to behavior modification protocols and avoid relapse behavior such as consuming the previously abused substance. In this example, the user device 108 can generate the physiologic data 124 and determine the social context 126, 128, and 130. The servers 122 uses the distances 126, 128, and 130 to generate the social-context data 132. The user device 104 gathers user-entered data 132, and the server 122 combines these three types of data. In some other configurations (not shown,) some or all of the activity performed by the servers 122 can be performed on other devices shown in the system 100. For example, user device 104 can perform some or all of the processing described, instead of sending the data to the servers 122.

When the user 106 is at risk of relapse behavior, the user device 104 is configured to generate an intervention alert 102 for the user's attention. In some cases, this intervention alert may be or include a simple red light that the user understands as an intervention alert 102.

For example, when the user's 106 physiologic data 124 indicates that the user 106 is in a craving state, and at the same time the user's 106 social-context data 132 indicates that the user is in the vicinity of a person the user has a history of drug use with, the user device 108 can alert the user through the use of vibration, emitting an audible alarm, etc. The user device 108 can then ask the user 106 if they are at risk of drug use. If the user 106 indicates they are at risk, the user device 104 can generate an intervention alert 102. In this example, the intervention alert can include a text communication to the user 106 to ask the user to engage in a Cognitive Behavior Therapy (CBT) exercise. In another case, the intervention alert 102 may automatically record the social context and transmit the record to a clinician, aiding the clinician in understanding the factors that influence the user's addiction, or place a phone call to the user's addiction sponsor or clinician so that this person can work with the user at this critical moment.

In another example, the user 106 may be a person diagnosed with Post-Traumatic Stress Disorder (PTSD,) which causes the user 106 to act out violently. In this example, when the user's physiologic data 102, social-context data 132, and user-entered data 134 indicate the user 106 is likely to enter violent state, the intervention alert 102 can be provided to a different user device of a different person. For example, the system can be configured to provide an alert to the user's 106 spouse. In such an example, the spouse may be shown as the user 114, and the intervention alert 102 may be provided to the user 114 by the user device 118 with a message to remove other people from the area so that they are not at risk of injury by the user 106.

In another example, the user 106 may be a child that is part of a family receiving family counseling. While the user 106's parents are engaging in counseling sessions, the data 124, 132, and 134 may be collected and analyzed in order to determine if the parents are having a positive or negative impact on the stress state of the user 106. If the behavior of a parent is having a detrimental impact on the user 106, the intervention alert can be generated and sent directly to a counselor, to instruct the user 106 to alert the counselor, or take another appropriate form given the medical needs of the user 106.

In another example, the user 106 may receive an intervention alert when they are within proximity of another person or place. For example, if the user 106 is dealing with anxiety in the workplace, the data 124, 132, and 134, along with location data, can be used to determine if an intervention alert 102 should be generated before the user arrives at work. A similar process may be used, for example, to provide an intervention alert 102 when a substance abuse patient nears or enters a liquor store or bar, etc.

While the alert 102 is being referred to as an intervention alert 102, it will be understood that the intervention alert 102 may be used for non-intervention purposes. For example, an intervention alert 102 may be used to send a user a quality-control questioner to help a user trouble-shoot a technical problem with software on their device, an information-gathering questioner to gather information that can be used in a future intervention, or to push emergency information to a user from, e.g., weather, traffic, or civil emergency services. For example, when to users 116 and 114 spend time in the same area and then leave, going separate ways, a questioner may be sent to one or both of the users 116 and/or 114.

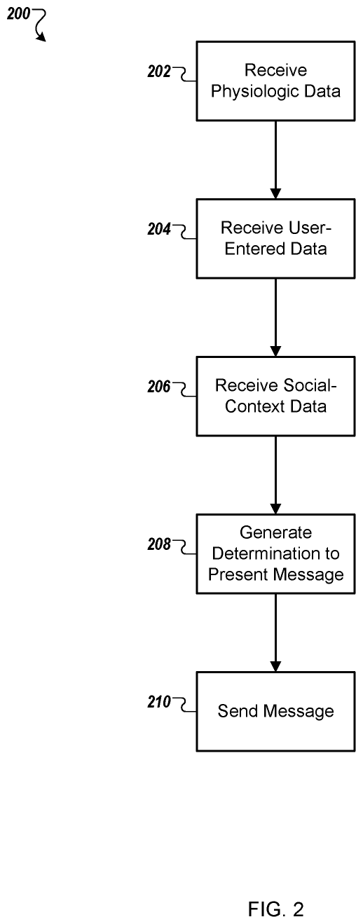

FIG. 2 shows a flowchart of an example process 200 for generating and delivering a message on a user device. The process 200 can be used with a variety of systems, including the system 100. However, other system or systems may be used to perform this or other similar processes.

Physiologic data generated from the sensing of one or more physiological parameters of a user is received 202. For example, the user device 108 can sense the user's 106 cardiac action, or the user device 104 can sense the walking steps taken by the user.

User-entered data for the user is received 204. For example, the user can press buttons or type in response to prompts on the user device 104.

Social-context data describing one or more other user within the immediate physical area of the user is received 206. For example, the server 122 can identify which of the users 110, 112, and 114 are within the social context of the user 106 based on the distances 126, 128, and 130.

A determination, from i) the physiologic data, ii) the user-entered data, and iii) the social-context data, that an automated message should be send to a user device is generated 208. For example, generating the determination that the automated message should be sent to a user device comprises: initially using at least one of the group consisting of i) the physiologic data, ii) the user-entered data, and iii) the social-context data to generate an initial determination that the user is in a particular state; and after generating the initial determination, using at least one of the group consisting of i) the physiologic data, ii) the user-entered data, and iii) the social-context data to confirm the initial the determination. That is to say, two of the types of data may be first used to identify an initial determination (e.g., increased heart-rate, near a liquor store,) and then a third type of data may be used to verify the initial determination (e.g., asking the user if they are going to the liquor store or just out for a jog.) Some predictors may also use simulated and/or estimation data as part of the prediction process. For example, a predictor may use multiple imputation, use a generalized adversarial network (GAN), to generate new cases from statistical information, use processes with Monte Carlo algorithms to fit a model, or use a forward-simulation engine to estimate implications of a choice.

Generating the determination that the automated message should be sent to a user device comprises applying i) the physiologic data, ii) the user-entered data, and iii) the social-context data to a predictor that generates a prediction of the user's particular state (e.g., current state, near-future state, far future state, recent historical state, far historical state). For example, one or more predictors may be created by a human expert, by heuristics, or by machine learning techniques. These predictors may use the data 124, 132, and 134 as input and output predictions of state along with confidence values. The outputs may be in the form of discrete outcomes (e.g., happy), continuous values (3.5743985467 points of happiness and 4.453 points of social tension), etc.

In some cases, a predictor may use human-user input as part of the prediction process. For example, a clinician or other user may review some or all of the i) the physiologic data, ii) the user-entered data, and iii) the social-context data to provide expert predictions to be used as input into the predictor. As will be understood, this expert prediction may be only one of multiple input signals used by the predictor, and may be mixed with non-user computations that are automatically performed on computer data.

The automated message is sent to the user device, responsive to the generating the determination 210. For example, the user device 102 can generate and audible tone to remind the user of their commitment to avoid liquor.

These messages can be sent to the user 106, to another user, or to a health care provider or similar support person. For example, the user 106 can receive real-time updates, while messages can be compiled into long-term trends for a clinician to review and act upon.

The automated messages can include only human-readable content, only machine instructions, or both. For example, human-readable content can include text, graphics, or sounds (or instructions to render these) that are comprehensible to a human. This can include text that is presented for a user to read, visual elements that are animated, sounds to be played, etc. Machine instructions can include text, data, or other instructions that are configured to cause a machine to activate in a particular way. This can include instructions to turn on a sensor (e.g., a video camera near the user) or turn off a device (e.g., turn off a TV is the programming is showing that it is creating a negative effect on the user.)

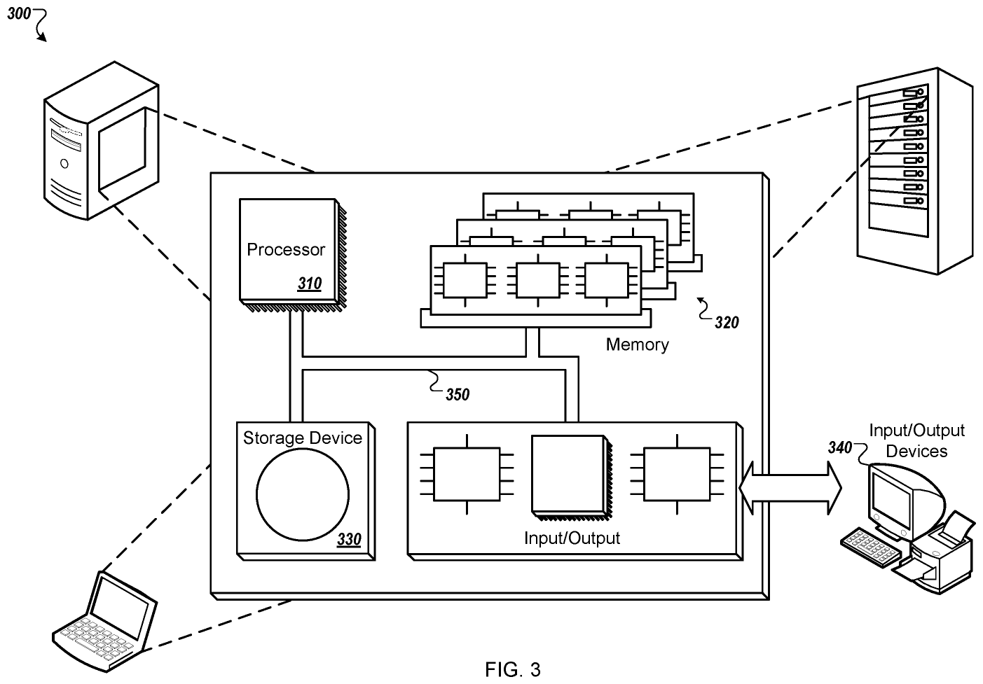

FIG. 3 is a schematic diagram that shows an example of a computing system 300. The computing system 300 can be used for some or all of the operations described previously, according to some implementations. The computing system 300 includes a processor 310, memory 320, a storage device 330, and an input/output device 340. Each of the processor 310, the memory 320, the storage device 330, and the input/output device 340 are interconnected using a system bus 350. The processor 310 is capable of processing instructions for execution within the computing system 300. In some implementations, the processor 310 is a single-threaded processor. In some implementations, the processor 310 is a multi-threaded processor. The processor 310 is capable of processing instructions stored in the memory 320 or on the storage device 330 to display graphical information for a user interface on the input/output device 340.

The memory 320 stores information within the computing system 300. In some implementations, the memory 320 is a computer-readable medium. In some implementations, the memory 320 is a volatile memory unit. In some implementations, the memory 320 is a non-volatile memory unit.

The storage device 330 is capable of providing mass storage for the computing system 300. In some implementations, the storage device 330 is a computer-readable medium. In various different implementations, the storage device 330 may be a floppy disk device, a hard disk device, an optical disk device, or a tape device.

The input/output device 340 provides input/output operations for the computing system 300. In some implementations, the input/output device 340 includes a keyboard and/or pointing device. In some implementations, the input/output device 340 includes a display unit for displaying graphical user interfaces.

Some features described can be implemented in digital electronic circuitry, or in computer hardware, firmware, software, or in combinations of them. The apparatus can be implemented in a computer program product tangibly embodied in an information carrier, e.g., in a machine-readable storage device, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM (erasable programmable read-only memory), EEPROM (electrically erasable programmable read-only memory), and flash memory devices; magnetic tape device, magnetic disks such as internal hard disks and removable disks; magneto-optical disks; and CD-ROM (compact disc read-only memory) and DVD-ROM (digital versatile disc read-only memory) disks. The processor and the memory can be supplemented by, or incorporated in, ASICs (application-specific integrated circuits).

To provide for interaction with a user, some features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

Some features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN (local area network), a WAN (wide area network), and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

What is claimed is:

1. A method for the automated generation of message on a user device, the method comprising:
receiving physiologic data generated from the sensing of one or more physiological parameters of a user;
receiving user-entered data for the user;
receiving social-context data describing one or more other user is within social communication with the user;
generating a determination, from i) the physiologic data, ii) the user-entered data, iii) the social-context data, iv) historical, archival, or prerecorded data about the individual or others that records at least one profile of historical data that an automated message should be sent to one or more user devices; and
sending, responsive to the generating the determination and to the one or more user devices, the automated message.

2. The method of claim 1, wherein generating the determination that the automated message should be sent to a user device comprises:
initially using at least one of the group consisting of i) the physiologic data, ii) the user-entered data, and iii) the social-context data to generate an initial determination that the user, dyad, or group is in a particular state; and
after generating the initial determination, using at least one of the group consisting of i) the physiologic data, ii) the user-entered data, and iii) the social-context data to confirm the initial the determination.

3. The method of claim 2, wherein generating the determination that the automated message should be sent to a user device comprises applying i) the physiologic data, ii) the user-entered data, and iii) the social-context data to a predictor that generates a prediction of the user's particular state.

4. The method of claim 1, wherein the physiologic data comprises readings of at least one of the group consisting of cardiac action, respiratory action, gross body-motion, body temperature, skin electrical properties, functional or structural brain signals.

5. The method of claim 1, wherein the social-context data is generated from location data comprises at least one of the group consisting of Global Positioning System (GPS) readings, geographic coordinates, data-network based geopositioning readings, and a proximity measure to a physical device.

6. The method of claim 1, wherein the social-context data is generated by identifying at least one of the group consisting of a location of other users, a social context of at least one other user, and a location of devices of other users.

7. The method of claim 6, wherein identifying at least one of the group consisting of the location of other users and the location of devices of other users comprises at least one of the group consisting of gathering data from a Bluetooth data connection, gathering data from a Zigbee data connection, gathering data from a Near Field Communication (NFC) data connection, gathering data from an audio sensor, gathering data from a Radio Frequency Identification (RFID) sensor, and gathering data from a sensor.

8. The method of claim 7, wherein the sensor is at least one of the group consisting of a microphone, a camera, a depth sensor, a thermal sensor, a vibration sensor, an appliance activity sensor, a standalone broadcast beacon and a receiver, a weight and pressure sensors, a light detecting and ranging (LIDAR) sensor, a sonar sensor, an ultrasonic sensor and a radio reflectance sensor.

9. The method of claim 1, wherein sending the automated message comprises at least one of the group consisting of sending the automated message to a device associated with the user, sending the automated message to another user within the social-context, sending the automated message to another user not within the social-context, ending the automated message to a device not associated with a user within the social-context, and storing the automated message for display at a later time.

10. The method of claim 1, where generating a determination that an automated message should be send to a user device further comprises using v) other theory-, evidence-, or other model-based rules, and vi) with human approval or adjustment.

11. The method of claim 1, the method further comprising including the sending of the automated message to a long-term report for use by another user.

12. A system for the automated generation of message on a user device, the system comprising:
one or more computer processors; and
non-transitory computer memory tangibly storing instructions that, when executed by the one or more processors, cause at least one of the processors to perform operations comprising:
receiving physiologic data generated from the sensing of one or more physiological parameters of a user;
receiving user-entered data for the user;
receiving social-context data describing one or more other user is within social communication with the user;
generating a determination, from i) the physiologic data, ii) the user-entered data, iii) the social-context data, iv) historical, archival, or prerecorded data about the individual or others that records at least one profile of historical data that an automated message should be sent to one or more user devices; and
sending, responsive to the generating the determination and to the one or more user devices, the automated message.

13. The system of claim 12, wherein generating the determination that the automated message should be sent to a user device comprises:
initially using at least one of the group consisting of i) the physiologic data, ii) the user-entered data, and iii) the social-context data to generate an initial determination that the user, dyad, or group is in a particular state; and
after generating the initial determination, using at least one of the group consisting of i) the physiologic data, ii) the user-entered data, and iii) the social-context data to confirm the initial the determination.

14. The system of claim 13, wherein generating the determination that the automated message should be sent to a user device comprises applying i) the physiologic data, ii) the user-entered data, and iii) the social-context data to a predictor that generates a prediction of the user's particular state.

15. The system of claim 12, wherein the physiologic data comprises readings of at least one of the group consisting of cardiac action, respiratory action, gross body-motion, body temperature, skin electrical properties, functional or structural brain signals.

16. The system of claim 12, wherein the social-context data is generated from location data comprises at least one of the group consisting of Global Positioning System (GPS) readings, geographic coordinates, data-network based geopositioning readings, and a proximity measure to a physical device.

17. The system of claim 12, wherein the social-context data is generated by identifying at least one of the group consisting of a location of other users, a social context of at least one other user, and a location of devices of other users.

18. The system of claim 17, wherein identifying at least one of the group consisting of the location of other users and the location of devices of other users comprises at least one of the group consisting of gathering data from a Bluetooth data connection, gathering data from a Zigbee data connection, gathering data from a Near Field Communication (NFC) data connection, gathering data from an audio sensor, gathering data from a Radio Frequency Identification (RFID) sensor, and gathering data from a sensor.

19. The system of claim 18, wherein the sensor is at least one of the group consisting of a microphone, a camera, a depth sensor, a thermal sensor, a vibration sensor, an appliance activity sensor, a standalone broadcast beacon and a receiver, a weight and pressure sensors, a light detecting and ranging (LIDAR) sensor, a sonar sensor, an ultrasonic sensor and a radio reflectance sensor.

20. A non-transitory computer-readable media tangibly storing instructions that, when executed by one or more processors, cause at least one of the processors to perform operations comprising:
receiving physiologic data generated from the sensing of one or more physiological parameters of a user;
receiving user-entered data for the user;
receiving social-context data describing one or more other user is within social communication with the user;
generating a determination, from i) the physiologic data, ii) the user-entered data, iii) the social-context data, iv) historical, archival, or prerecorded data about the individual or others that records at least one profile of historical data that an automated message should be sent to one or more user devices; and
sending, responsive to the generating the determination and to the one or more user devices, the automated message.
